(19) United States
(12) Patent Application Publication
(10) Pub. No.: US 2014/0329917 A1
(43) Pub. Date: Nov. 6, 2014

(54) APPARATUS, SYSTEM AND METHOD FOR IDENTIFYING CIRCULATING TUMOR CELLS

(71) Applicants: Gerd Marienfeld, Lauda-Konigshofen (DE); Peter Kuhn, Solana Beach, CA (US); Anand Kolatkar, San Diego, CA (US); Dena Marrinucci, San Diego, CA (US)

(72) Inventors: Gerd Marienfeld, Lauda-Konigshofen (DE); Peter Kuhn, Solana Beach, CA (US); Anand Kolatkar, San Diego, CA (US); Dena Marrinucci, San Diego, CA (US)

(21) Appl. No.: 14/363,290

(22) PCT Filed: Dec. 7, 2012
(86) PCT No.: PCT/US12/68586

§ 371 Date: Jun. 5, 2014

Related U.S. Application Data
(60) Provisional application No. 61/568,793, filed on Dec. 9, 2011.

(51) Int. Cl. G01N 33/574; G01N 33/483; G01N 33/50; B01L 3/00

(52) U.S. Cl. CPC G01N 33/57492; B01L 3/5088; G01N 33/4833; G01N 33/5011
USPC 514/789; 435/289.1; 435/288.7; 435/29; 435/34; 435/7.23; 506/9; 435/6.14; 435/6.12; 435/287.2

(57) ABSTRACT

Apparatus, systems and methods are provided for the identification of various objects, particularly circulating tumor cells. In one aspect the system includes, but is not limited to, a scanning system, an image storage system, and an analysis system. The analysis system preferably identifies desired objects, such as complete cells, based on various criteria, which may include cell nuclear area or volume, CD-45 negative status, and cytokeratine positive status. Preferably included is a slide for containing the cells during the imaging step, the well including a planar bottom surface, a border at the periphery of the well defining sides for the well, the border being adjacent the bottom surface of the well and providing a fluidic seal there between. The invention herein provides for a single imaging well, providing for substantially a monolayer of objects, e.g., cells.

### BACKGROUND OF THE INVENTION

1. Field of the Invention

The invention relates generally to medical diagnostics and more specifically to identifying and categorizing circulating tumor cells (CTCs).

2. Background Information

The field of circulating tumor cell research has evolved rapidly in response to a significant unmet medical need for longitudinal disease monitoring in patients with epithelial cancers (carcinomas). Predicting and monitoring therapy response and disease progression are particularly important due to changes in the therapy-responsiveness of disease over the course of a patient's cancer. In liquid tumors such as leukemia, the malignant cells can be easily sampled from the bloodstream at many points during the disease, and appropriate therapy adjustments applied. However, solid tumors such as carcinomas are generally sampled only at the time of initial diagnosis, as tissue biopsies are invasive procedures with known risks. Occasionally, a repeat tumor sampling is collected at the time when distant metastasis first becomes apparent, to confirm that distant lesions in fact represent metastases from the patient's known primary tumor.

In current understanding of cancer behavior, while progress has been made in understanding the solid tissue forms of primary and metastatic carcinomas in their respective microenvironments, a substantial gap exists in understanding carcinoma behavior during the fluid phase, wherein it occupies and spreads via the bloodstream. For cancers that occur predominantly as solids, the elusive and scant circulating component contains within it the cells giving rise to future distant metastases, and as such, represents a compelling target for invention.

Research to fully characterize the clinical significance of this fluid phase of solid tumors has been hindered by the lack of easily accessible and reliable experimental tools for the identification of circulating tumor cells (CTCs). The unknown character and low frequency of CTCs in the blood, combined with the difficulty of distinguishing between cancerous versus normal epithelial cells, has significantly impeded research into how the fluid phase might be clinically important. The ideal fluid phase biopsy would find significant numbers of CTCs in most epithelial cancer patients, and would preserve and present CTCs to a diagnostic pathologist and/or researcher in a format that enables not only enumeration but further molecular, morphologic and/or phenotypic analysis. In addition, it should preserve all other CTC-like cell populations within the sample for further analysis.

Currently, the only FDA-approved technology for CTC detection is based on immunomagnetic enrichment. This current “gold standard” test is called CellSearch® and employs an immunomagnetic enrichment step to isolate cells that express the epithelial cells adhesion molecule (EpCAM) [1]. Additionally, to be identified as a CTC, the cell must contain a nucleus, express cytoplasmic cytokeratin, and have a diameter larger than five microns. This technology has uncovered the prognostic utility of enumerating and monitoring CTC counts in patients with metastatic breast, prostate, and colorectal cancers; however, the sensitivity of this system is low, finding no or few CTCs in most patients [2, 3]. Most follow-on CTC technologies have reported higher sensitivity and are pursuing variations of the enrichment strategy; however, these approaches directly bias the detectable events towards those that have sufficient expression of the protein selected for the initial enrichment step [4-8].

Pitfalls abound in the field of CTC biology. Among the most difficult issues are sensitivity and specificity. The rate of true biologic positives in cancer patients is unknown, and the rate of circulating benign epithelial cells in healthy people or people with non-malignant disease, is likewise not known with certainty. For sensitivity—a positive test in the presence of disease (in this case, the biologic presence of CTCs)—the strategy commonly used in place of sure knowledge is either spiking experiments placing cell line cells into whole blood, or else published data from other researchers using technologies that may vary significantly. Both approaches are problematic and the issue persists in the field. Specificity—a negative test in the absence of disease—can be addressed at least partially by evaluating patient samples that, according to our current understanding of cancer, should be negative for circulating cancer cells.

To this end, healthy donor blood is generally used as a negative control, and while published numbers vary, in general only low numbers of CTCs are found in clinically healthy people. For the results discussed herein, the healthy donor population consists of non-laboratory member volunteers of varying ages who are not known to have cancer but who have not undergone extensive medical evaluations for occult cancers. Since all cancer is, of course, initially asymptomatic, finding a truly negative population for specificity determination would be an expensive and long term endeavor, as one would need to conduct either invasive medical testing on the apparently healthy subjects, or else wait a sufficient amount of time to be sure they do not manifest some type of carcinoma over subsequent years.

Various formats have been used in the prior art to present patient samples for assays. Those formats have included fluidic systems, such as flow cytometry based systems, and static systems. FIG. 1 shows a plan view of a three well plate as used in prior art static imaging systems. A slide includes three equally sized well regions. By way of example, each well region 10 is a 1.45 cm square. The resulting total well area for the slide is 6.3 cm². The periphery of the wells is 17.4 cm. The percentage of the overall slide occupied by the three wells is 23.6%. An estimated number of cells per slide is in the range from 1.25 to 1.5 million cells.

FIG. 2 shows a plan view of a twelve well plate as used in the prior art. A slide includes twelve equally sized well regions 12. By way of example, each well region is a circle with a 0.5 cm diameter. The resulting total well area for the slide is 2.4 cm². The periphery of the wells is 19 cm. Finally, the percentage of the overall slide occupied by the three wells is 12.8%. An estimated number of cells per slide is in the range from 500 to 600 thousand cells.

Despite intensive effort, the detection of CTCs has heretofore remained challenging. What is required is a sensitive, specific system which can efficiently, expeditiously and inexpensively detect CTCs.

### SUMMARY OF THE INVENTION

Apparatus, system and method are provided for the identification of various objects, particularly CTCs.

As such, in one aspect, the invention provides a system for assaying cells. The system includes a well of the present invention, an illumination system, an imaging system, an analysis module having functionality for analyzing cell selection criteria, and a user output. In embodiments system may include, but is not limited to, a scanning system, an image storage system, and an analysis system The analysis system preferably identifies desired objects, such as complete cells, based on various criteria, which may include cell nuclear area or volume, CD-45 negative status, and cytokeratine positive status. Preferably included is a slide bearing a well for containing the cells during the imaging step, the well including a planar bottom surface, a border at the periphery of the well defining sides for the well, the border being adjacent the bottom surface of the well and providing a fluidic seal there between.

In another aspect, the invention provides a well for assaying cells which is disposed on a surface of a substrate. The well includes a planar bottom surface, and a border forming a periphery of the well, the border being adjacent the bottom surface and providing a fluidic seal therebetween. Embodiments of the invention provide for a single imaging well, providing for substantially a monolayer of objects, e.g., cells. The well has an area preferably greater than 7.5 cm², more preferably greater than 10 cm², and most preferably substantially 11.7 cm². The perimeter of the well is preferably substantially 12.5 cm, more preferably substantially 14.5 cm and most preferably substantially 15.7 cm, correspondingly. The percentage of the top surface of the slide covered by the well is preferably substantially 40%, more preferably 53% and most preferably substantially 62%. The wells are sized so as to permit the imaging of a monolayer of preferably 1.6 to 1.9 million cells, more preferably 2.1 to 2.6 million cells, and most preferably, 2.5 to 3 million cells, respectively. The preferred imaging wells have a total of four sides. By reducing the number of sides (as compared to the prior art, for example, the 3 well slide has 12 sides) and their perimeter, the edge effects associated with the side wall boundary are minimized.

In one implementation, the approach used herein to identify CTCs in high definition (HD-CTCs) is distinct in that it does not rely on any single protein enrichment strategies. Instead, all nucleated cells are retained and immunofluorescently stained with monoclonal antibodies targeting cytokeratin (CK), an intermediate filament found exclusively in epithelial cells, a pan leukocyte specific antibody targeting CD45, and a nuclear stain, DAPI. The nucleated blood cells are imaged in multiple fluorescent channels to produce high quality and high resolution digital images that retain fine cytologic details of nuclear contour and cytoplasmic distribution. This enrichment-free strategy results in high sensitivity and high specificity, while adding high definition cytomorphology to enable detailed morphologic characterization of a CTC population known to be heterogeneous. An advantage of this approach is that multiple analysis parameters can be pursued to identify and characterize specific populations of interest.

Embodiments of the instant inventions have been used to assay samples using “HD-CTCs” in metastatic cancer patients. The key innovative aspects of this assay are its simplicity, with minimal processing to the blood specimen, and its ability to enable professional morphologic interpretation with diagnostic pathology/cytopathology quality imagery.

In yet another aspect, the invention provides a method for performing a cellular assay. The method includes contacting a sample having a population of cells with the well of the present invention, and analyzing the population of cells via the system of the present invention, thereby preforming the cellular assay. In embodiments, the analysis includes characterization of cell types within the population of cells, such as CTCs.

In yet another aspect, the invention provides a method of detecting a CTC in a sample. The method includes contacting the well of the present invention with the sample, analyzing the population of cells via the system of the present invention; and detecting a CTC based on the analysis, thereby detecting a CTC in the sample. In embodiments, more than 2, 5, 7, 10, 15, 20 or 50 circulating tumor cells are detected per ml of sample.

In yet another aspect, the invention provides a method for diagnosing cancer or providing a prognosis for cancer in a subject. The method includes contacting a well of the present invention with a sample including a population of cells from the subject, analyzing the population of cells via the system of the present invention, detecting a CTC in the cell population, characterizing the CTC, and determining a diagnosis or prognosis based on the characterization, thereby diagnosing or providing a prognosis for cancer in the subject.

In yet another aspect, the invention provides a method for determining responsiveness of a subject to a chemotherapeutic regime. The method includes contacting the well of the present invention with a sample including a population of cells from a subject, analyzing the population of cells via the system of the present invention, detecting a CTC via the analysis, and characterizing the CTC to determine efficacy of administration of a chemotherapeutic agent, thereby determining responsiveness of the subject to the therapeutic regime.

In yet another embodiment the invention provides a kit. The kit includes at least one well of the present invention, reagents for immunologically determining the presence of cytokeratin or CD45 in a cell, and instructions for utilizing the kit the detect a CTC in a sample.

The apparatus, systems and methods described herein demonstrate the first use of the HD-CTC assay in a controlled prospective protocol which addressed the reliability and robustness of the assay, compares sensitivity in a split sample comparison with the Cellsearch® assay, and establishes the incidence of HD-CTCs and HD-CTC clusters in patients with metastatic breast, prostate, and pancreatic cancers as well as normal controls. Importantly, the definition of “HD-CTC” preferably requires one or more of the requirements that the cell(s) have an intact nucleus, express cytokeratin and not CD45, be morphologically distinct from surrounding benign white blood cells (WBCs), and have cytologic features consistent with intact morphologically abnormal epithelial cells suitable for downstream analysis.

### BRIEF DESCRIPTION OF THE DRAWINGS

FIG. 1 is a plan view of a prior art 3 well plate.

FIG. 2 is a plan view of a prior art 12 well plate.

FIG. 3 is a functional block diagram of the overall HD CTC system.

FIG. 4 is a component level diagram of the scanning and imaging components of the system.

FIG. 5 shows possible image slices through the measured parameter space, representing filtering of cell based on these parameters.

FIG. 6 is a plan view of a representative slide and well.

FIG. 7 is a perspective view of a representative slide and well.

FIG. 8 shows the mean observed SKBR3s plotted against expected SKBR3s.

FIG. 9 shows a comparison of CTC counts between two separate processors on 9 different cancer patient samples. CTC/mL counts ranged from 0 to 203.

FIG. 10 shows comparative test data of the systems, apparatus and methods described here, versus the CellSearch® product.

FIG. 11 shows test results graphing the quantity of CTCs for various samples, for prostate, pancreatic, breast tumors, and a comparison to healthy population.

FIG. 12 shows the quantity of CTCs for various patient samples relative to breast cancer.

FIG. 13 shows the normalized nuclear area versus nuclear area for white blood cells (WBCs) and CTCs, including a blow-up of the base-line region.

### DETAILED DESCRIPTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In general, reference to “a circulating tumor cell” is intended to refer to a single cell, while reference to “circulating tumor cells” or “cluster of circulating tumor cells” is intended to refer to more than one cell. However, one of skill in the art would understand that reference to “circulating tumor cells” is intended to include a population of circulating tumor cells including one or more circulating tumor cells.

The term “circulating tumor cell” (CTC) or CTC “cluster” is intended to mean any cancer cell or cluster of cancer cells that is found in a subject's sample. Typically CTCs have been exfoliated from a solid tumor. As such, CTCs are often epithelial cells shed from solid tumors found in very low concentrations in the circulation of patients with advanced cancers. CTCs may also be mesothelial from sarcomas or melanocytes from melanomas. CTCs may also be cells originating from a primary, secondary, or tertiary tumor. CTCs may also be circulating cancer stem cells. While the term “circulating tumor cell” (CTC) or CTC “cluster” includes cancer cells, it also is intended to include non-tumor cells that are not commonly found in circulation, for example, circulating epithelial or endothelial cells. Accordingly tumor cells and non-tumor epithelial cells are encompassed within the definition of CTCs.

The term “cancer” as used herein, includes a variety of cancer types which are well known in the art, including but not limited to, dysplasias, hyperplasias, solid tumors and hematopoietic cancers. Many types of cancers are known to metastasize and shed circulating tumor cells or be metastatic, for example, a secondary cancer resulting from a primary cancer that has metastasized. Additional cancers may include, but are not limited to, the following organs or systems: brain, cardiac, lung, gastrointestinal, genitourinary tract, liver, bone, nervous system, gynecological, hematologic, skin, breast, and adrenal glands. Additional types of cancer cells include gliomas (Schwannoma, glioblastoma, astrocytoma), neuroblastoma, pheochromocytoma, paraganlioma, meningioma, adrenalcortical carcinoma, medulloblastoma, rhabdomyoscarcoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia; and skin cancers including malignant melanoma, basal cell carcinoma, squamous cell carcinoma, Karposi's sarcoma, moles dysplastic nevi, lipoma, angioma, dermatofibroma, keloids, sarcomas such as fibrosarcoma or hemangiosarcoma, and melanoma.

Using the apparatus and methods described herein, CTCs may be detected and characterized from any suitable sample type. As used herein, the term “sample” refers to any sample suitable for the methods provided by the present invention. The sample may be any sample that includes rare cells suitable for detection. Sources of samples include whole blood, bone marrow, pleural fluid, peritoneal fluid, central spinal fluid, urine, saliva and bronchial washes. In one aspect, the sample is a blood sample, including, for example, whole blood or any fraction or component thereof. A blood sample, suitable for use with the present invention may be extracted from any source known that includes blood cells or components thereof, such as veinous, arterial, peripheral, tissue, cord, and the like. For example, a sample may be obtained and processed using well known and routine clinical methods (e.g., procedures for drawing and processing whole blood). In an embodiment, an exemplary sample may be peripheral blood drawn from a subject with cancer.

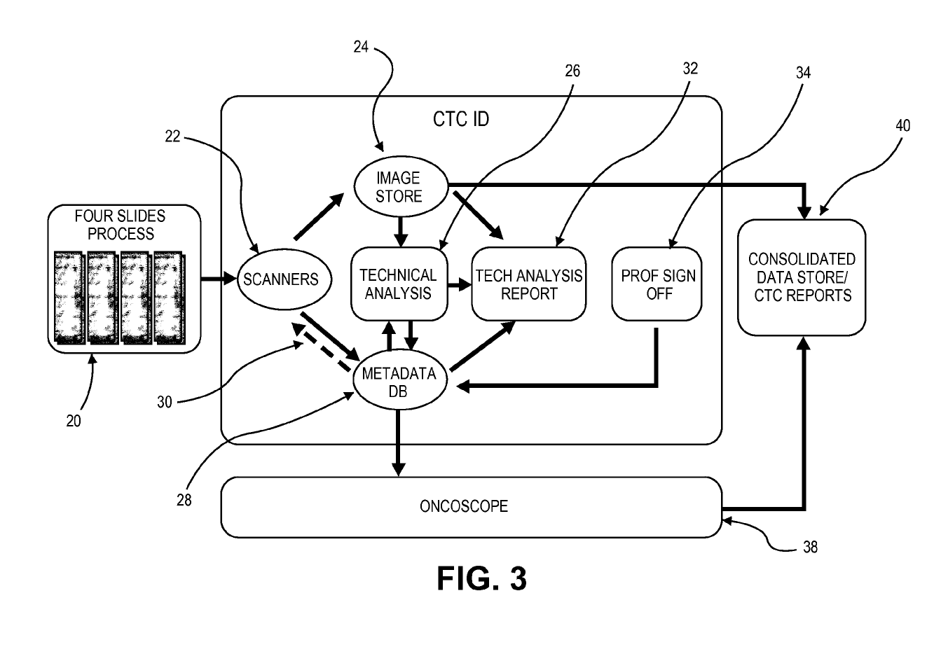

FIG. 3 is a functional block diagram of the overall system. One or more slides 20 are prepared for analysis. See the detailed description, below, for sample collection, preparation and processing description. Scanners 22 image the one or more slides 20. The scanners 22 preferably are multi-channel scanners, such as 4 color scanners. Data from the scanners 22 is supplied to an image store 24. The image store 24 may be composed of storage, preferably mass storage, such as RAID systems, as are known to those skilled in the art. The scanned data from the image store is provided to one or more of the technical analysis module 26, the technical analysis report module 32 and or the output 34 to a professional review, such as by a specialist, for review. The technical analysis module 26 serves at least to analyze the data from the image store 24 in ways described in detail, below. The analysis includes, but is not limited to, analyzing the cell for nuclear area or size (such as by analyzing for the intensity of blue DAPI), analyzing for the absence of CD-45 (such as by scanning for the intensity of the secondary antibody associated with CD-45 antibody, and/or analyzing for the intensity of the antibody associated with cytokeratine. Preferably, the technical analysis report comprises an HTML file with data, including cell or object images, in the file. Automated analysis may be supplemented with analysis by a medical professional.

The output of the technical analysis module 26 is preferably provided to a medadata database 28. The metadata database includes the information generated by all of the various forms of analysis of the data. A return loop control path 30 permits the use of the scan data and analysis to then control the re-imaging of the slide(s) 20. In the event that further analysis of an object, such as a cell, is required, the system can cause the reimaging of the object. The degree of adhesion or adherence of the objects to the slide should be sufficient to maintain the location of the objects on the slide for at least the duration of the reimaging. In yet a longer timeframe, the degree of adhesion or adherence of the objections to the slide should be sufficient to permit the subsequent identification of specific objects identified by the analysis, such as described below, for the subsequent further processing of on object, such as for genotyping or other subsequent analysis. The length of time for storage of the slides, and the desire that the cell location remain stationary, may range from hours to months. Preferably, the system includes a consolidated store 40 such as for the data and reports.

Information from the metadata database 28 is preferably provided to an data inventory management system 38. The system comprises the management system for the overall system. Among other data, the system 38 maintains the correlation of patient identification with the slides.

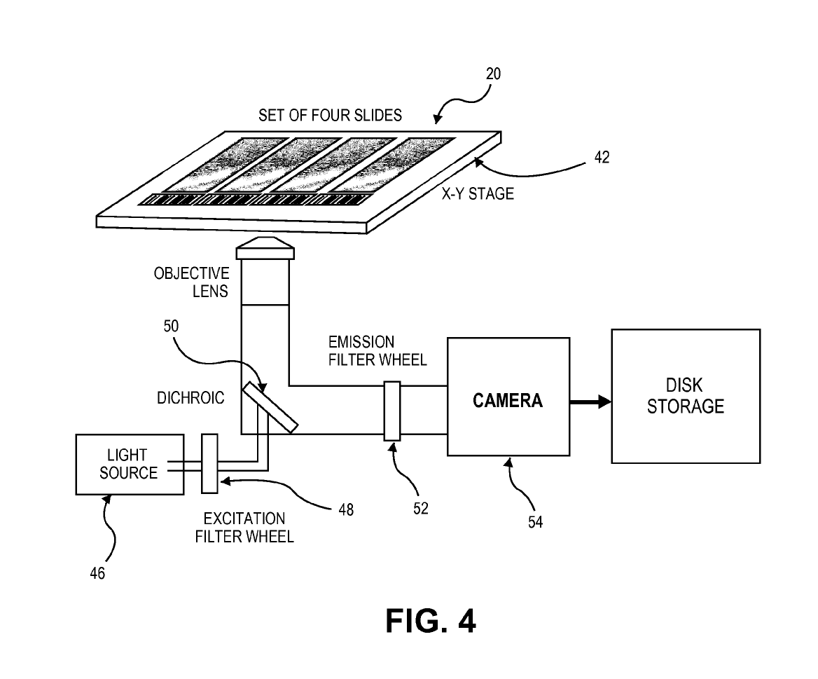

FIG. 4 is a schematic view of one possible implementation of the scanning system. A stage 42, such as an x-y stage, supports one or more slides 20. For the size of well area described herein, four slides permit the scanning of approximately 10 million cells, which is a typical size sample for one patient. The optical path may take any form consistent with the embodiments described herein, The illumination components may include light source 46, and optional excitation filter wheel 48. The light source is preferably a broad spectrum illuminator. A dichroic mirror 50 serves to pass the illumination to the slides, and to permit the return illumination to the camera 54. An optional emission filter wheel 52 may be placed between the mirror 50 and camera 54. The output is then stored as described above.

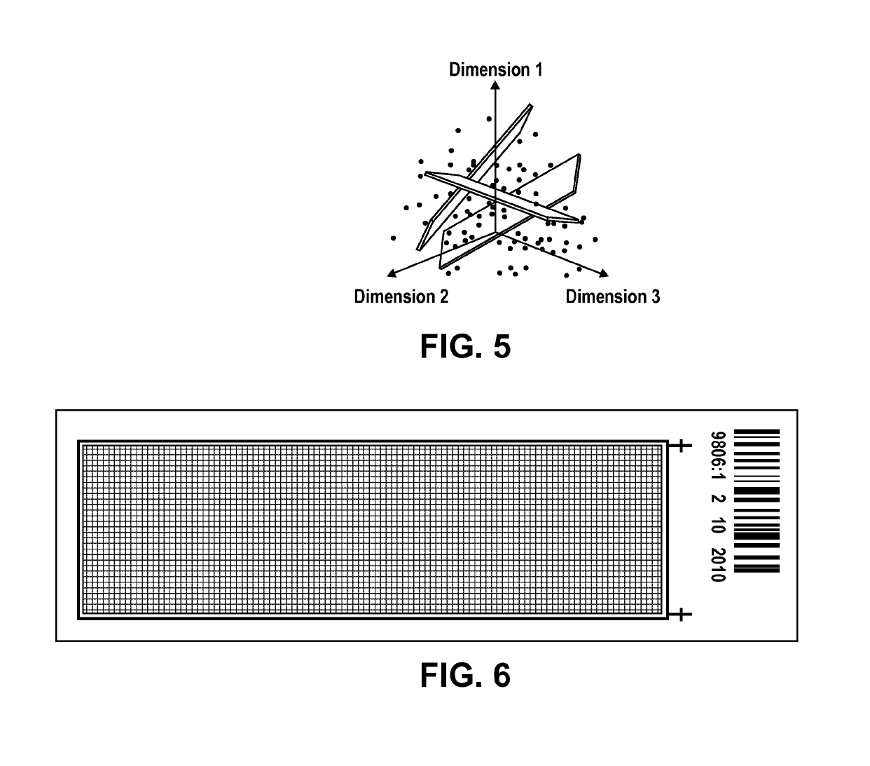

FIG. 5 shows various options for the scanning of objects supported by the slide 20. Scanning and imaging may be in multiple dimensions, preferably in a three-dimensional frame work. Preferably, the objects are disposed on the slide in a monolayer which is sufficiently flat to permit the scanning and imaging of the objects in an efficient manner, preferably being disposed within a single focal plane. The planar or flat slide permits the imaging of the monlayer in a consistent manner as the deviation of the image plane is minimized. Imaging on a flat surface also enables easier Z-stacking of images. As shown in FIG. 5, the imaging planes may be at various orientations, which may be in a non-planar relationship. As shown in FIG. 5, detection of CTC candidate cells typically relies on several parameters measured from the slide imagery. For example, dimensions 1-3 could be nuclear area, cytokeratine intensity and CD-45 intensity. The planes in FIG. 5 represent the cut-off limits for each of the measured parameters that define CTC candidates. Alternatively or additionally, light field camera systems may be utilized in which a digital camera includes light sensors which capture light rays even beyond a single focal plane, permitting the software assembly of images from various image planes. Lenses, such as microlenses, may be used in association with the digital light sensors.

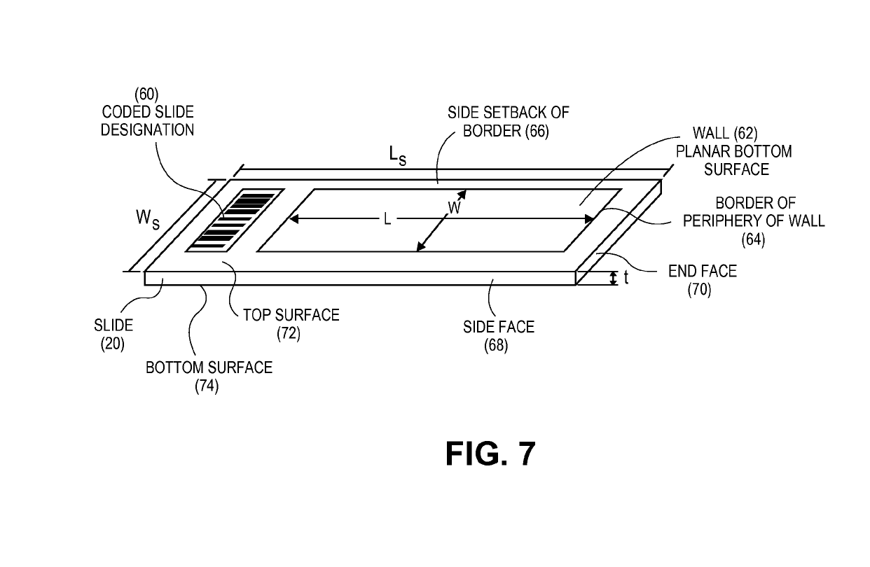

FIGS. 6 and 7 define various particulars of the slide 20. The slide may be of any size or geometry consistent with the embodiments of the inventions described, herein. In one implementation, the slide 20 is generally rectangular in shape, with a length Lₛ, a width Wₛ, and a thickness t. Representative dimensions are L, of substantially 7.5 cm, Wₛ of substantially 2.5 cm, and thickness t of 7 mm. The slide 20 preferably includes a top surface 72, a parallel bottom surface 74, side setbacks 66, side faces 68 and end faces 70. A slide identification 60 may be provided, such as via a barcode. Such slides are available from various sources, including Marienfeld Laboratory Glassware.

A well 62 is provided to contain and maintain the materials to be imaged. In the format described in detail herein, the well 62 is rectangular and has a length L and a width W. Representative inner dimensions for the well 62 may be, by way of example L of substantially 5.85 cm and W of substantially 2.5 cm. The periphery or perimeter of the well 62 may be defined by a border 64, whether a specific structural border or by other materials, such as by a border of hydrophobic material. The border may also be termed a boundary. The border or boundary is adapted to receive and contain the cell suspension and all other reagents, solutions, buffers or other liquids that are used in the process. The border or boundary in combination with the top side of the slide form the well. With these dimensions, the area of the well 62 is substantially 11.7 cm² and the perimeter of the well 62 is substantially 15.7 cm. The degree of setback of the well 62 from the edges of the slide 20 may be set based on other aspects of the system, such as the particulars of the scanning system. One embodiment provides for a single imaging well 62, providing for substantially a monolayer of objects, e.g., cells, to be imaged, having an area preferably greater than 7.5 cm², more preferably greater than 10 cm², and most preferably substantially 11.7 cm². The perimeter of the well 62 is preferably substantially 12.5 cm, more preferably substantially 14.5 cm and most preferably substantially 15.7 cm, correspondingly. The percentage of the top surface of the slide covered by the well is preferably substantially 40%, more preferably 53% and most preferably substantially 62%.

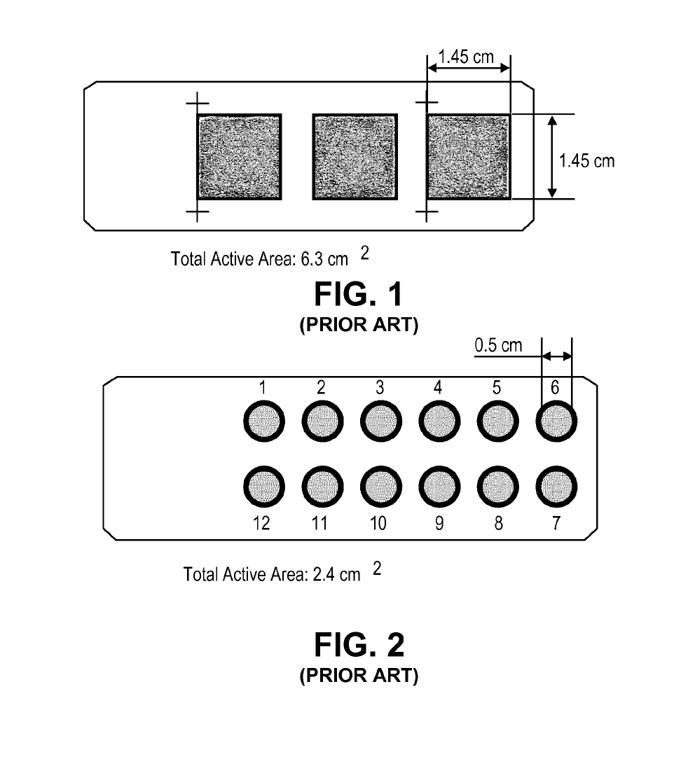

The preferred imaging wells 62 have a total of four sides. By reducing the number of sides (as compared to the prior art, for example, the 3 well slide has 12 sides (see FIG. 1)) and their perimeter, the edge effects associated with the side wall boundary are minimized. The wells are sized so as to permit the imaging of a monolayer of preferably 1.6 to 1.9 million cells, more preferably 2.1 to 2.6 million cells, and most preferably, 2.5 to 3 million cells, respectively.

To summarize, the following parameters define various measures of the systems, apparatus and methods, when imaging cells in a substantially monolayer in the well described herein:

| Area | Perimeter | % Coverage | Number of Cells |
|---|---|---|---|
| 11.7 cm² | 15.7 cm | 62% | 2.5 to 3 million |
| 10 cm² | 14.5 cm | 53% | 2.1 to 2.6 million |
| 7.5 cm² | 12.5 cm | 40% | 1.6 to 1.9 million |

The slide may optionally be provided with reference marks. If the cells are sufficiently fixed, in location, other structures may be used for indexing the slide. By way of example, the border may be utilized, more particularly, the 90 degree angle at the corner of the well may be used for reference.

The apparatus and systems described herein provide for maximized and optimized cell density and minimized edge effects. In the preferred embodiment, a single cell well is utilized to hold at least 1.5 million cells, an optionally even more, such as 3 million cells. In the preferred embodiment, four side walls serve to contain that cell population. In contrast, use of three field-slides instead of a single field slide would require use of two to three times as many slides and deal with twelve (3×4) edges on each slide instead of just four edges. Any fluid distribution suffers from edge effects no matter how hydrophobic the edges are. Cell distribution shows that the cell density at the edges goes down significantly. While a standard sized microscope slide can be used, it is not so limited. Larger glass slides may be utilized consistent with the goals of the embodiments described herein. However, using a conventional sized slide results in process benefits by staying with a standard size, for which a large base of installed machines exist, such as automation, existing microscope systems and storage systems, to name a few.

The system further preferably includes a single cover slide per slide. The system serves to optimize speed while producing sufficient quality. By preferably avoiding non-flat surfaces, stacked cells, changing fluid thicknesses and or using multiple coverslides on each slide, both the imaging setup and the data collection speed and quality is increased. A single and very flat homogenous monolayer is preferred. Yet a further advantage of using a single cover slide is that a uniform surface is presented for imaging. A much more even mounting media distribution is provided using a single cover slip instead of three as would be needed in a standard three well slide.

In embodiments, a sample processed as described herein includes greater than about 1, 2, 5, 7, 10, 15, 20, 30, 40, 50, 100, 200, 300, 400, 500, 600, 700, 800, 900, or even 1000 rare cells or CTCs.

While the methods described in this invention are useful in detecting CTCs, as discussed throughout, the invention also is useful in characterization of CTCs. In particular, use of the various combinations of detectable markers and computational methods for performing cell imaging and analysis allow for meaningful characterization useful in assessing cancer prognosis and in monitoring therapeutic efficacy for early detection of treatment failure that may lead to disease relapse. In addition, CTC analysis according to the invention enables the detection of early relapse in presymptomatic patients who have completed a course of therapy. This is possible because the presence of CTCs has been associated and/or correlated with tumor progression and spread, poor response to therapy, relapse of disease, and/or decreased survival over a period of time. Thus, enumeration and characterization of CTCs provides methods to stratify patients for baseline characteristics that predict initial risk and subsequent risk based upon response to therapy.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Accordingly, in another embodiment, the invention provides a method for diagnosing or prognosing cancer in a subject. The method includes detecting CTCs as described herein. CTCs may then be analyzed to diagnose or prognose cancer in the subject. As such, the methods of the present invention may be used, for example, to evaluate cancer patients and those at risk for cancer. In any of the methods of diagnosis or prognosis described herein, either the presence or the absence of one or more indicators of cancer, such as, a cancer cell, or of any other disorder, may be used to generate a diagnosis or prognosis.

In one aspect, a blood sample is drawn from the patient and processed to detect CTCs as described herein. Using the method of the invention, the number of CTCs in the blood sample is determined and the CTCs are characterized by analysis of the detectable markers and other data gathered from imaging the cells. For example, analysis may be performed to determine the number and characterization of CTCs in the sample, and from this measurement, the number of CTCs present in the initial blood sample may be determined.

In various aspects, analysis of a subject's CTC number and characterization may be made over a particular time course in various intervals to assess a subject's progression and pathology. For example, analysis may be performed at regular intervals such as one day, two days, three days, one week, two weeks, one month, two months, three months, six months, or one year, in order to track level and characterization of circulating epithelial cells as a function of time. In the case of existing cancer patients, this provides a useful indication of the progression of the disease and assists medical practitioners in making appropriate therapeutic choices based on the increase, decrease, or lack of change in circulating epithelial cells, such as the presence of CTCs in the patient's bloodstream. Any increase, be it 2-fold, 5-fold, 10-fold or higher, in the number of CTCs over time decreases the patient's prognosis and is an early indicator that the patient should change therapy. Similarly, any increase, be it 2-fold, 5-fold, 10-fold or higher, indicates that a patient should undergo further testing such as imaging to further assess prognosis and response to therapy. Any decrease, be it 2-fold, 5-fold, 10-fold or higher, in the number of CTCs over time shows disease stabilization and a patient's response to therapy, and is an indicator to not change therapy. For those at risk of cancer, a sudden increase in the number of CTCs detected may provide an early warning that the patient has developed a tumor thus providing an early diagnosis. In one embodiment, the detection of revealed CTCs increases the staging of the cancer.

In any of the methods provided herein, additional analysis may also be performed to characterize CTCs, to provide additional clinical assessment. For example, in addition to image analysis, gene expression analysis and PCR techniques may be employed, such as gene chip analysis and multiplexing with primers specific for particular cancer markers to obtain information such as the type of tumor, from which the CTCs originated, metastatic state, and degree of malignancy. Additionally, cell size, DNA or RNA analysis, proteome analysis, or metabolome analysis may be performed as a means of assessing additional information regarding characterization of the patient's cancer. In various aspects, analysis includes antibodies directed to or PCR multiplexing using primers specific for one or more of the following markers: EGFR, HER2, ERCC1, CXCR4, EpCAM, E-Cadherin, Mucin-1, Cytokeratin, PSA, PSMA, RRM1, Androgen Receptor, Estrogen Receptor, Progesterone Receptor, IGF1, cMET, EML4, or Leukocyte Associated Receptor (LAR).

For example, the additional analysis may provide data sufficient to make determinations of responsiveness of a subject to a particular therapeutic regime, or for determining the effectiveness of a candidate agent in the treatment of cancer. Accordingly, the present invention provides a method of determining responsiveness of a subject to a particular therapeutic regime or determining the effectiveness of a candidate agent in the treatment of cancer by detecting CTCs of the subject as described herein and analyzing the detected CTCs. For example, once a drug treatment is administered to a patient, it is possible to determine the efficacy of the drug treatment using the methods of the invention. For example, a sample taken from the patient before the drug treatment, as well as one or more cellular samples taken from the patient concurrently with or subsequent to the drug treatment, may be processed using the methods of the invention. By comparing the results of the analysis of each processed sample, one may determine the efficacy of the drug treatment or the responsiveness of the patient to the agent. In this manner, early identification may be made of failed compounds or early validation may be made of promising compounds.

Four important indicators that provide insight to the clinical activity of candidate compounds include HER2, EGFR, CXCR4, and EphB4 RTK. HER2 provides an indicator of malignancy of a cell by determining mRNA stability and subcellular localization of HER2 transcripts. The resistance of EGFR to acquire mutations, and/or the mutations acquired provides important indicators of the activity of a candidate compound in addition to possible alternative compounds that may be used in combination with the candidate compound. An assessment of the level of DNA repair interference induced with platinum provides insight as to the status of the CXCR4 marker and metastatic condition. Additionally, assessment of the status of EphB4 receptor tyrosine kinase provides insight as to the metastatic potential of the cell. Accordingly, using the methods of the present invention, patients taking such candidate drugs may be monitored by taking frequent samples of blood and determining the number of circulating epithelial cells, for example CTCs, in each sample as a function of time. A further analysis of the Her2, EGFR, CXCR4, and EphB4 RTK indicators provides information as to pathology of the cancer and efficacy of the candidate drug. Similarly, ERRC1, Cytokeratin, PSA, PSMA, RRM1, Androgen Receptor, Estrogen Receptor, Progesterone Receptor, IGF1, cMET, EML4 and others provide insight into the clinical activity of candidate compounds. The analysis of these indicators of clinical activity may be through analysis of detectable markers as discussed herein (e.g., immunohistochemistry and fluorescent in situ hybridization (FISH)) or further analysis via techniques such as sequencing, genotyping, gene expression or other molecular analytical technique.

Analysis of CTCs provide a method of determining candidate subjects for a particular clinical trial. For example, the detected CTCs of a candidate may be analyzed to determine whether specific markers exist in order to determine whether the particular therapeutic regime of the clinical trial may be potentially successful. Accordingly in another embodiment, the invention provides a method for determining a candidate subject for a clinical trial. The method includes detecting CTCs of the subject as described herein. The CTCs may then be analyzed to determine whether the candidate subject is suitable for the particular clinical trial.

Analysis of CTCs during a clinical trial will provide information on whether the patient is responding or not responding to the experimental drug, where no substantial change or a decrease in revealed CTCs indicates response and an increase in revealed CTCs indicates poor response. The increase or decrease may be 2-fold, 10-fold or higher. This information is an early indicator of the drug's effectiveness and may be used by the investigators as a secondary endpoint in the clinical trial.

The following example is intended to illustrate but not limit the invention.

### Example 1

### Detection and Characterization of CTCs

Experimental Results

CTC to HD-CTC: Definitional Refinements.

The system preferably defines one or more measures for the cells to be utilized in the analysis. Various approaches define an intact CTC based on the investigation of large numbers of candidate events in patients with epithelial cancers, with direct comparison to cell from the solid forms of the same tumor in the same patient [9-12]. Based on these definitions, and utilizing the HD-CTC assay to refine criteria, a definition of an HD-CTC was established. This definition has been developed to ensure that an HD-CTC is a cell that has the highest potential of being an intact cell originating from a solid deposit of carcinoma in the patient's body. All other populations that partially fulfill these requirements but fall short of the strict inclusion criteria discussed below are tracked in the analysis since many of them likely represent fragmented or apoptotic tumor cells that might have biologic significance [13], but are excluded in the HD-CTC count because of their unpredictable suitability for evaluation by further downstream methodologies. For purposes of this application, various criteria meeting some or all of these may be used.

CTCs may be morphologically characterized and credentialed in relation to their primary tumors, in case studies of breast, colorectal, and lung cancer patients. Through morphologic examination of CTCs in a patient with a well differentiated lung adenocarcinoma, circulating cells were identified with morphologic features consistent with this type of tumor, including, for example, cells with relatively low nuclear-to-cytoplasmic ratios. The morphology of the tumor cells identified in circulation mimicked the morphology found in that patient's fine needle aspirate biopsy of the primary tumor [9]. Evaluated in a larger cohort of breast and colorectal cancer patients the CTCs exhibited a high degree of inter- and intra-patient heterogeneity in cytologic appearance consistent with the morphologic heterogeneity of cells commonly found in the primary and metastatic tumor [10, 11].

This platform allows for simultaneous cytomorphologic review of fluorescent images with individual channel images, augmented with cell-by-cell annotation with ancillary semi-quantitative data regarding size and fluorescent intensity of objects. HD-CTCs are classified as cells that are a) cytokeratin positive: b) CD45 negative, with an intact non-apoptotic appearing nucleus by DAPI imaging. Positivity for CK is defined as the fluorescent signal being significantly above the signal of surrounding cells. Negativity is defined as being the same level or below the signal of the surrounding cells. Negativity for CD45 is defined as having intensity below visual detection under the boundary condition that 99% of all cells are detectable globally. A gallery of representative HD-CTCs found in cancer patients is not shown, however, HD-CTCs are cytokeratin positive, CD45 negative, contains a DAPI nucleus, and are morphologically distinct from surrounding white blood cells.

Mild apoptotic changes in the cytoplasm are accepted, such as cytoplasmic blebbing visualized in the cytokeratin channel as long as the nucleus does not appear apoptotic. In addition to these semi-quantitative characteristics HD-CTCs must be morphologically distinct from normal WBCs, and must have a morphology that is compatible with a malignant cell by criteria used in standard diagnostic cytopathology, predominantly embodied as enlarged size, but also encompassing cytomorphologic features such as architectural organization of nucleus and cytoplasm, cytoplasmic shape, and nuclear shape. A lower nuclear size cut-off of 1.3 times the mean WBC nuclear size may be set. Although somewhat arbitrary, as no modern consensus has been established and the biologic truth is as yet unknown, this limit is set based on evaluation of the largest nuclear size of cells identified as WBCs in healthy donors showing false nonspecific staining with cytokeratin (i.e. CD45 positive and cytokeratin positive). As virtually all viable epithelial cells are larger than virtually all leukocytes in routinely fixed and stained human tissues across the clinical spectrum of cancer diagnosis, this approach is felt to be a conservative assumption. At the other end of the spectrum, a common morphologic feature of HD-CTCs is larger nuclei up to five times the average size of surrounding WBC nuclei. Other commonly observed features include nuclear contours distinct from those of surrounding WBC nuclei (e.g. elongation), large cytoplasmic domain with an eccentric distribution of cytoplasm relative to nucleus, polygonal or elongated cytoplasmic shape, and frequent doublets and clusters of 3 or more HD-CTCs.

TABLE 1
Percentage of patients with HD-CTCs per 1 mL of blood
obtained from metastatic prostate, breast, and pancreatic
cancer patients as well as normal controls.

| | N | ≧2 | ≧5 | ≧10 | ≧50 |
|---|---|---|---|---|---|
| Prostate | 20 | 90% | 80% | 65% | 40% |
| Breast | 30 | 80% | 70% | 60% | 27% |
| Pancreatic | 18 | 61% | 44% | 44% | 11% |
| Normal | 15 | 7% | 0% | 0% | 0% |

Incidence of HD-CTCs in Patients with Metastatic Cancer.

HD-CTCs were enumerated in a cohort of 30 metastatic breast cancer patients, 20 metastatic prostate cancer patients, 18 metastatic pancreatic cancer patients, and 15 normal controls. The incidence of HD-CTCs in the three types of cancers investigated is displayed in Table 1. Using this approach, ≧5 HD-CTCs/mL were found in 80% of the prostate cancer patients (mean=92.2), 70% of the breast cancer patients (mean=56.8), 50% of the pancreatic cancer patients (mean=15.8), and 0% of normal controls (mean=0.6).

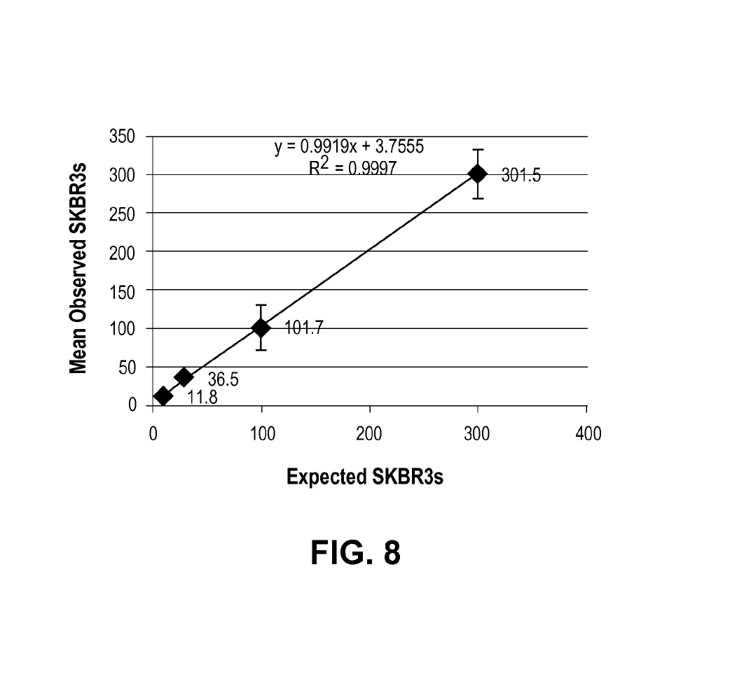

FIG. 8 shows mean observed SKBR3s plotted against expected SKBR3s. Four aliquots of normal control blood was spiked with varying numbers of SKBR2 cells to produce 4 slides with approximately 10, 30, 100, and 300 cancer cells per slide. The mean of each quadruplicate is displayed as well as error bars noting standard deviation.

Assay Linearity and Sensitivity Using Spike-In Experiments.

To test assay linearity and sensitivity, various numbers of breast cancer cell line SKBR3s were spiked into normal control blood in quadruplicates and processed according to the HD-CTC assay. As displayed in FIG. 8, mean observed SKBR3s is plotted against expected SKBR3s and displays a correlation coefficient (R²) of 0.9997.

Assay Robustness of HD-CTC Counts in Patients with Carcinomas.

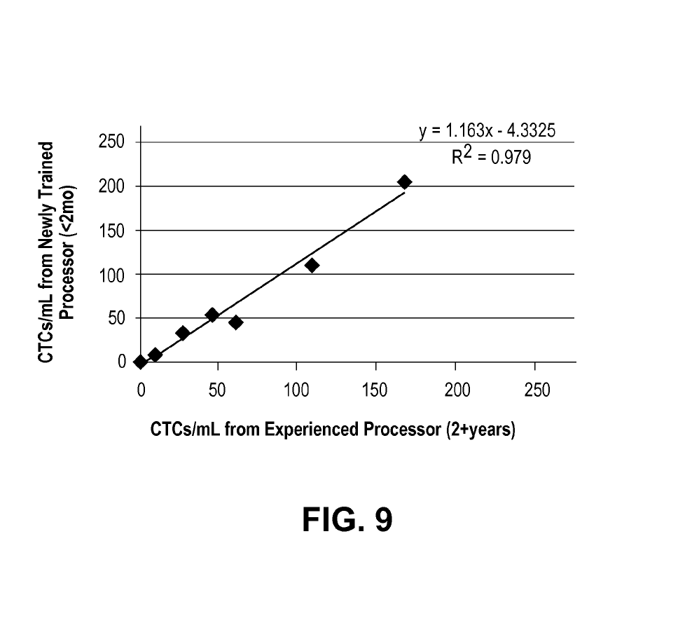

Assay robustness of the I-ID-CTC assay was tested against multiple processors and split samples. Duplicate tests were performed by two separate processors on 9 different patient samples. FIG. 9 displays a comparison of HD-CTC/mL counts between two processors using split samples giving a regression equation for this comparison of Y=1.163×-4.3325 with a correlation coefficient (R²) of 0.979. All data were analyzed by a single operator blinded to the experiment. The slope of the line is greater than one suggesting a slight systematic linear variation between the two processors.

FIG. 9 is a comparison of CTC counts between two separate processors on 9 different cancer patient samples. CTC/mL counts ranged from 0 to 203.

Assay Specificity in Samples Front Normal Controls.

Fifteen healthy donors from an institutional healthy donor pool were evaluated as a control population consisting of 8 females and 7 males with an age range of 24 to 62 years. In all but one healthy control, the number of such events when corrected for volume was 1 HD-CTC/ml or less. The outlier was a healthy female donor with an HD-CTC count of 4/ml. Upon explicit re-review of each of her cells, about one third of them easily met all inclusion criteria, while the remaining two thirds fulfilled all criteria but were near the lower limit for inclusion by one or more criteria. Four other healthy donors fell into the non-zero category, with 1 HD-CTC/ml each. Explicit re-review of these cells revealed a similar pattern, in that about one third strongly met all criteria, while the remaining two thirds of the cells fulfilled criteria, but were near the lower limit for inclusion by one or more criteria. Examples of the latter type of event include cells that measure 30% larger than surrounding WBCs but do not appear significantly larger by morphologic evaluation, and cells that are slightly out of focus and might have apoptotic nuclear changes that are not detectable by eye, and finally, occasional cells that have objective cytokeratin intensity measurements above the cutoff but subjectively don't appear significantly brighter than surrounding WBCs by single channel fluorescent review.

Comparison of HD-CTC Assay to CellSearch®.

A total of 15 patients (5 metastatic breast cancer and 10 metastatic prostate cancer) were evaluated for CTCs with both Cellsearch® and the HD-CTC assays. Two tubes of blood were collected from each patient. One tube of 7.5 mL of blood was collected in CellSave tubes (Veridex, Raritan N.J.) and sent to Quest Diagnostics (San Juan Capistrano, Calif.) for enumeration of CTCs using the Cellsearch® assay. A second tube of blood was collected from each patient and processed according to the HD-CTC protocol 24 hours after the blood draw, consistent with the standard HD-CTC process in order to mimic the timing at which samples were processed at Quest Diagnostics. Table 2 shows the results from this side by side comparison. The CellSearch® assay detected 2 or more CTCs per 7.5 mL of blood in 5/15 patients tested. In contrast, the HD-CTC assay detected significantly higher numbers of CTCs in significantly more patients (HD-CTCs were identified in 14/15 patients tested).

TABLE 2
Comparison of HD-CTC Assay to CellSearch ® from
5 metastatic breast cancer patients and 10 metastatic prostate
cancer patients. CellSearch ® values are
extrapolated to number of CTCs per mL of blood.

| Cancer Type | HD-CTCs/mL | CellSearch/mL |
|---|---|---|
| Breast #1 | 49.3 | 0.1 |
| Breast #2 | 87 | 0 |
| Breast #3 | 33.4 | 0.1 |
| Breast #4 | 199.3 | 0.1 |
| Breast #5 | 5 | 3.1 |
| Prostate #1 | 2.3 | 0 |
| Prostate #2 | 8.4 | 0.4 |
| Prostate #3 | 107.3 | 2.8 |
| Prostate #4 | 1.3 | 0 |
| Prostate #5 | 150.5 | 0.1 |
| Prostate #6 | 0 | 0 |
| Prostate #7 | 1.4 | 0.5 |
| Prostate #8 | 1.5 | 0.1 |
| Prostate #9 | 145.3 | 0.8 |
| Prostate #10 | 57.6 | 0 |

Morphologic Range of HD-CTCs.

A morphologically heterogeneous population of HD-CTCs was found within and across patients. HD-CTCs had various shapes, sizes, and cytokeratin intensities. In some cases, distinctive cytologic features such as large size or polygonal cytoplasmic shape were quite distinctive and monotonous within the patient's sample. In other cases, there was cytomorphologic variability between HD-CTCs within a single sample. Cell size also varied: many patient samples had HD-CTCs with nuclei uniformly three or four times the size of neighboring WBC nuclei, while other patients had cells with nuclei uniformly only 1.3 times the size of neighboring WBC nuclei. Some patients had a range of sizes. A lower limit criterion was selected for HD-CTC nuclear size of 1.3 times the average WBC nucleus, based on evaluation of the largest nuclear size of cells identified as WBCs showing false nonspecific staining with cytokeratin (i.e. CD45 positive and cytokeratin positive).

Using this platform allows for detailed morphologic evaluation, HD-CTC doublets and clusters were identified in the majority of the cancer patients in this cohort (88%), ranging from clusters of 2 HD-CTCs to greater than 30 HD-CTCs (data not shown).

Clusters were found in most patients with cancer. Clusters ranged from 2 to over 30 HD-CTCs. Each HD-CTC was determined to be cytokeratin positive, CD45 negative, contain a DAPI nucleus, and was morphologically distinct from surrounding nucleated cells.

Morphology of ‘Other’ Cell Types.

Other cell-like objects that are cytokeratin positive, CD45 negative, and contain a nucleus but do not meet the inclusion criteria, are not counted as HD-CTCs but are tracked by the assay. The purpose of this approach is to have strict inclusion/exclusion criteria for a specific intact phenotype of CTCs, while retaining access to objects that only partially fulfill such criteria, yet might still be clinically meaningful, such as apoptotic tumor cells, tumor cell fragments, or cells undergoing epithelial to mesenchymal transition [13].

Thus, in addition to tracking HD-CTCs, a number of different categories of cytokeratin positive cells were catalogued in this cohort of patients, including cells that had nuclei displaying apoptosis, cells that did not have circumferential cytokeratin, cells that were the same size or smaller than surrounding WBC, and cells that were cytokeratin dim or negative (data not shown). Finally, in addition to various types of bright cytokeratin positive cells, many patients had a substantial number of cells with nuclei that were morphologically distinct from surrounding WBC, resembled the nuclei of the HD-CTCs within that sample, and were CD45 negative, but were also cytokeratin dim or negative (data not shown).

Some candidate HD-CTCs were excluded because they lacked various morphologic or morphometric inclusion criteria. For example, observed cytokeratin intensity was too dim; the nuclear size was too small; cytokeratin was observed to be insufficiently circumferential (surrounds less than 2/3 of nucleus); observed cytokeratin was too dim, even though cluster appeared to be of multiple large cells; nucleus showed apoptotic disintegration changes; nucleus was too small and cytoplasm was insufficiently circumferential; appeared to be a cell in late apoptosis; nucleus was too small (same size as surrounding WBC nuclei); cytokeratin was present, but not circumferential; and cytoplasm was insufficiently circumferential, and the nucleus was too small.

Various types of suspected CTCs were also found in a single prostate cancer patient. For example, some were negative for Cytokeratin and CD45, but had a nucleus that was large and looked like other HD-CTCs found in this patient. Typical HD-CTC were also observed in which the cells were cytokeratin positive, CD45 negative, with a DAPI nucleus. Clusters of HD-CTCs of multiple cells, e.g., 4 cells were also observed.

In light of the extensive current debate about the possible existence of carcinoma cells undergoing epithelial-to-mesenchymal transition, the appearance and protein expression pattern of these cells identifies them as possible candidates for such a cell type. Alternatively, these cells could be older tumor cells that have been stripped of most of their cytoplasm.

Fluid biopsy analysis holds the promise of revealing metastasis in action. Within this elusive cell population are the cancerous seeds that spread through the bloodstream and lead to eventual distant metastases. But in order to interrogate them and apply the findings clinically, the cells must be reliably recoverable in the majority of cancer patients. The apparatus, systems and methods described here yield a maximally inclusive, minimally destructive, yet cytologically selective platform that yields high quality cells in high definition in high numbers of cancer patients. Initially noted as a rare occurrence in prostate cancer patients [13], for the first time identifying clusters of these cells in the majority (88%) of patients with metastatic cancer.

The incidence of CTCs using the assay is much higher than that reported with many technologies and is in the same range as reported by the CTC-chip [6]. Additionally, a direct comparison to CellSearch® showed significantly more CTCs detected by the HD-CTC assay, in a higher proportion of patients. In addition to higher sensitivity, the assay also demonstrates robust performance in both cell lines and patient samples. The reproducibility and robustness of the assay with a semi-quantitative characterization of each event is critical for downstream analysis of cells.

Morphologically, a heterogeneous population of CTCs was found within and across patients. CTCs had various shapes, sizes, and cytokeratin intensities. In some cases, distinctive cytologic features such as large size or polygonal cytoplasmic shape were quite distinctive and monotonous within the patient's sample. In other cases, there was cytomorphologic variability between HD-CTCs within a single sample. Cell size also varied inconsistently; many patient samples had HD-CTCs with nuclei uniformly three or four times the size of neighboring WBC nuclei, other patients had cells with nuclei only a third again as large as neighboring WBC nuclei, and other patients had high intra-patient size variability.

Surprisingly, the cohort of patients demonstrated the common occurrence of clusters of HD-CTCs in most patients. 88% of the metastatic cancer patients evaluated in this cohort study showed clusters ranging in size from 2-30 HD-CTCs. Multiple questions arise around the presence of such clusters, including the rheology of transit through the circulatory system of such a large aggregate, as well as biological questions about whether such clusters represent ‘tumorlets’ that are transporting their own microenvironmental stroma with them as they travel and thus may be the most, or only, truly metastable circulating tumor cells. Current investigations are underway to further characterize the clusters in this cohort of patients.

In addition to enumerating HD-CTCs, various other categories of CTC-like cells were independently tracked, including cells that had nuclei displaying apoptosis, cells lacking circumferential cytokeratin, cells that were the same size or smaller than surrounding WBC, and CD45-negative cells that were cytokeratin dim or negative (data not shown). Although many of these events may in fact represent circulating malignant epithelial cells in various stages of biologic anoikis or mechanical disruption secondary to even the minimal processing utilized in the platform, others likely represent false positives of various types. An initial goal is to identify a population of cells with a very high likelihood of including all potentially metastasizing epithelial cells that are suitable for downstream analysis by secondary methodologies. Fragmented, disrupted, pyknotic or otherwise damaged carcinoma cells are not considered reliable for secondary analysis in standard diagnostic pathology, and thus they were excluded for purposes of ‘counting viable circulating tumor cells’ in this fluid phase biopsy platform as well. The systems, apparatus and methods of these embodiments locate, enumerate and track them, as it is recognized that their presence likely correlates overall with the tumor biology in the patient, either by reflecting overall tumor burden or by reflecting some as yet ill-understood complex equation involving tumor burden and tumor vascularity and efficiency of intravascular immune surveillance.

One of the interesting non-HD-CTC categories, often seen in patients who have HD-CTCs elsewhere on the slide, consists of cells with nuclei that are morphologically distinct from surrounding WBC, generally by size criteria, and were CD45 negative, but are also cytokeratin dim or negative. As one of the most significant advantages of the HD-CTC assay is that parallel aliquots of cells are frozen, allowing for retrospective marker selection in specific high-yield patient samples, ongoing studies to further characterize such cells is in progress. Possibilities include epithelial cells with denatured or stripped cytoplasm, cells aberrantly expressing or aberrantly lacking proteins typical for their biologic origin, or possibly cells undergoing a metaplastic process such as epithelial to mesenchymal transition. The assay and imaging platforms are currently limited to analysis of fixed cells; however efforts are underway to establish the potential utility of this approach for live cell enumeration and imaging.

While the sample sizes of the respective patient cohorts are still too modest to draw any firm conclusions, it is noteworthy that the frequency of detection, and relative concentration, of CTCs among different tumor types using the approach (prostate>breast>pancreatic) parallels the findings observed using other methods such as CellSearch®. Previous investigators have suggested that biologic or anatomic differences in tumor vascularization, the anatomic sites of metastasis, and whether tumor cells are filtered via the portal circulation may account for some of these differences [1].

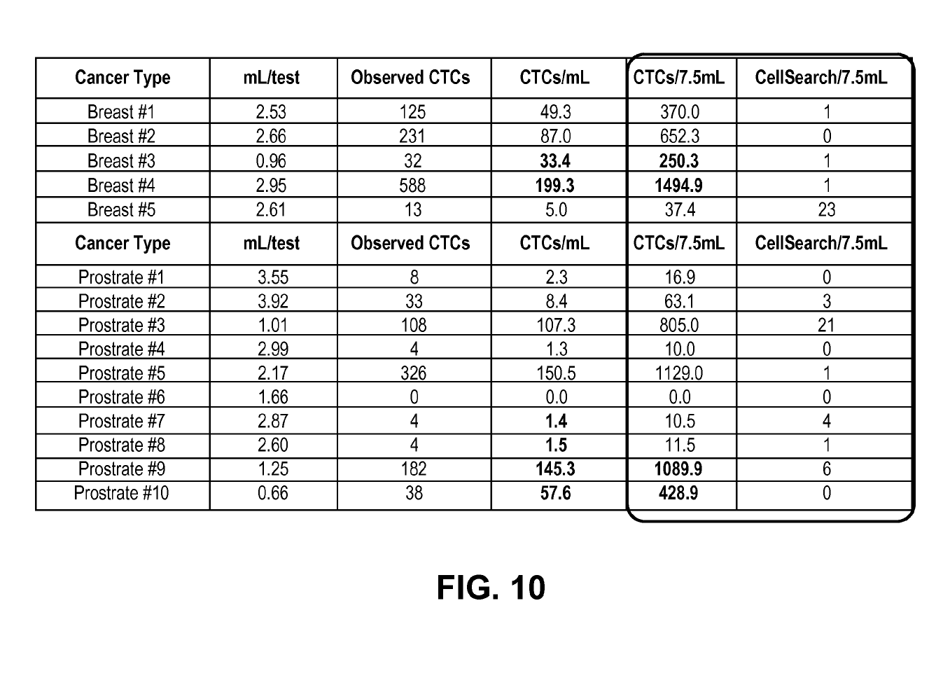

FIG. 10 shows comparative test data of the systems, apparatus and methods described here, versus the CellSearch® product. The left most column identifies five breast cancer tumors and for 10 prostate cancer tumors. The second column states the mL/test. The third column shows the observed CTCs using the systems, apparatus and methods of the described embodiment. The fourth column provides the calculated CTCs/mL. The two right-most columns provide the comparative data for the CellSearch® product, reported as per 7.5 ml (as compared to per mL in the fourth column.)

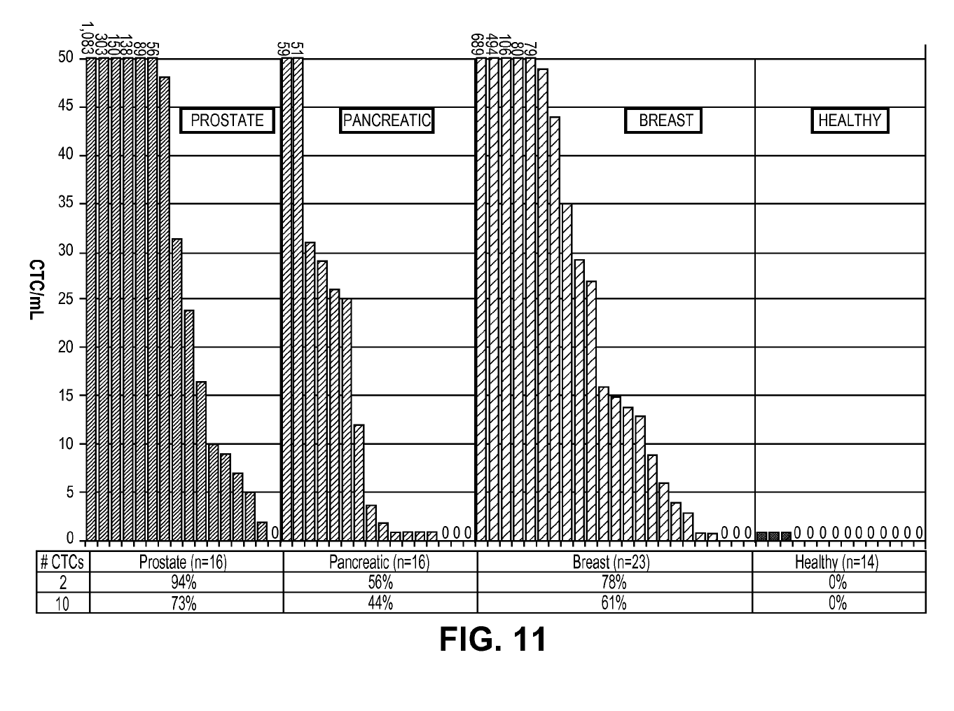

FIG. 11 shows test results graphing the quantity of CTCs for various samples, for prostate, pancreatic, breast tumors, and a comparison to healthy population. From left to right are data for prostate cancer, pancreatic cancer, breast cancer and for a presumed healthy population. These results provide the number of CTCs/ml by sample observed CTCs using the systems, apparatus and methods of the embodiments described herein.

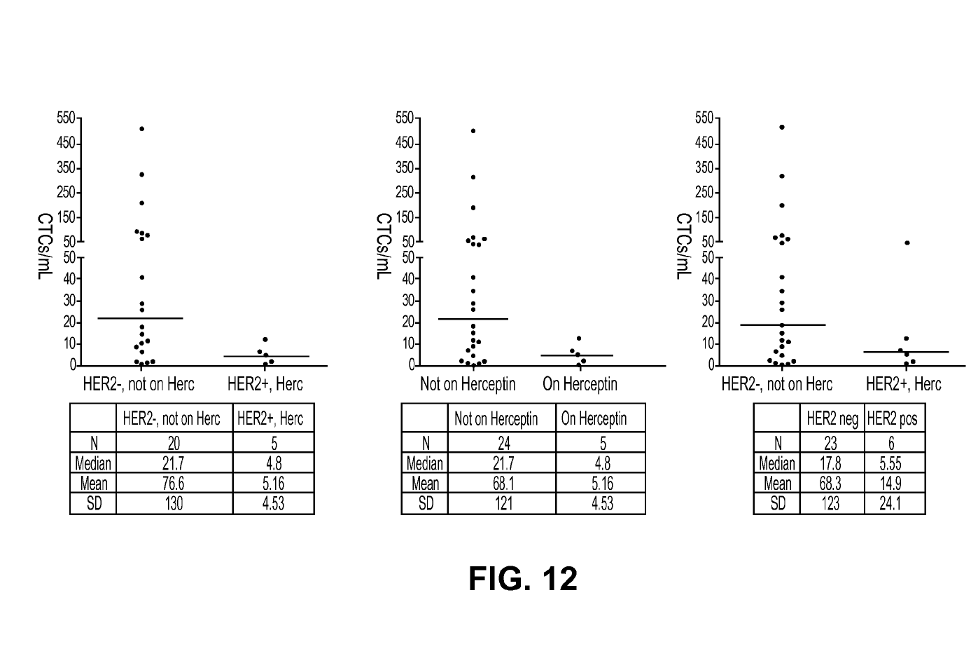

FIG. 12 shows the quantity of CTCs for various patient samples relative to breast cancer. The left-most graph shows HER2−, not on Herceptin and HER2+, Herceptin. The center graph shows a comparison of not on Herceptin (left) with on Herceptin (right). The right-most chart shows HER2 negative (left) versus HER2 positive (right)

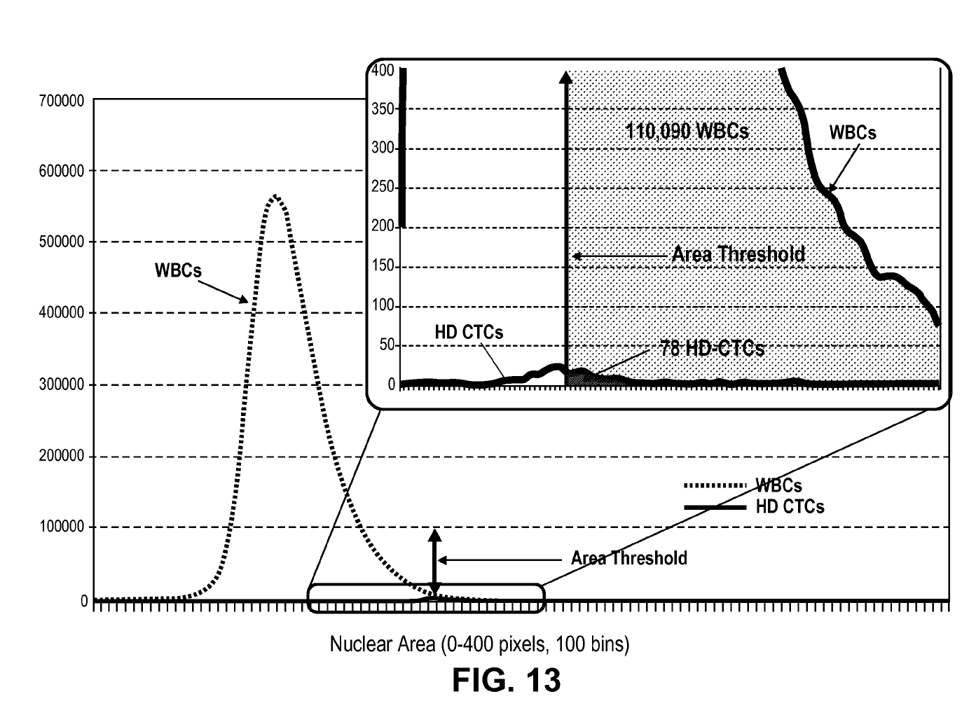

FIG. 13 shows the normalized nuclear area versus nuclear area for white blood cells (WBCs) and CTCs, including a blow-up of the base-line region. The left axis in the underlying graph is from 0 to 700,000. The blow-up is from 0 to 400. The benefit of use of multi-parameter analysis is supported by FIGS. 16 and 17. As shown in FIG. 13, a single parameter such as nuclear area may not reduce the number of candidates on a slide to a tractable amount. While is may appear that the use of a lower limit on nuclear size would remove most of the noise (WBCs), the blow-up shows that the number of non-CTC candidates is still large compared to the actual HD-CTCs. The use of more parameters, such as CK intensity and CD-45 intensity, serves to effectively filter out the non-HD CTC events.

In summary the HD-CTC assay (i) finds significant number of CTCs in most patients with metastatic cancer, (ii) has improved sensitivity over the Cellsearch® system. (iii) provides HD-CTCs in an ideal format for downstream characterization, (iii), enables the prospective collection of samples that can be stored frozen for long periods of time and then retrospectively analyzed as new assays or markers become available.

Experimental Methods

Patients and Blood Sample Collection.

Samples were collected from metastatic cancer patients in anti-coagulated blood tubes at Scripps Clinic. University of California, San Diego, Billings Clinic, and University of California, San Francisco under Institutional Review Boards (IRB) approved protocols. Samples from non-local sites (UCSF, Billings Clinic) were shipped overnight so that the sample was received and processed within 24 hours. Samples from local sites (Scripps Clinic and UCSD) were held at room temperature for 16-24 hours to mimic samples coming from non-local sites. Blood specimens were also drawn from normal controls from the The Scripps Research Institute (“TSRI”) Normal Blood Donor Service.

Blood Sample Processing for HD-CTC Detection.

Blood specimens were rocked for five (5) minutes before a white blood cell (WBC) count was measured using the Hemocue white blood cell system (HemoCue, Sweden). Based upon the WBC count, a volume of blood was subjected to erythrocyte lysis (ammonium chloride solution). After centrifugation, nucleated cells were re-suspended in Phosphate Buffered Saline (PBS) and attached as a monolayer on custom made glass slides. The glass slides are the same size as standard microscopy slides but have a coating that allows maximal retention of live cells. (A type of adhesion slides may be obtained at least from Marienfeld Laboratory Glassware (Germany)). Each slide can hold approximately 3 million nucleated cells, thus the number of cells plated per slide depended on the patients WBC count.

For HD-CTC detection in cancer patients for this study, four (4) slides were used as a test. The remaining slides created for each patient were stored at −80° C. for future experiments. Four slides were thawed from each patient. The cells were fixed with 2% paraformaldehyde, permeabilized with cold methanol, and non-specific binding sites were blocked with goat serum. Slides were subsequently incubated with monoclonal anti-pan cytokeratin antibody (Sigma) and CD45-Alexa 647 (Serotec) for 40 minutes at 37° C. After PBS washes, slides were incubated with Alexa Fluor 555 goat anti-mouse antibody (Invitrogen) for 20 minutes at 37° C. Cells were counterstained with DAPI for 10 minutes and mounted with an aqueous mounting media.

Imaging and Technical Analysis.

All four (4) slides from each patient were scanned using a custom made fluorescent scanning microscope which has been developed and optimized for fast, reliable scanning. Each slide was scanned entirely using a 10 OX objective lens in three (3) colors and produced over 6900 images. The resulting images were fed to an analysis algorithm that identifies likely candidate HD-CTCs based upon numerous measures, including cytokeratin intensity, CD45 intensity, as well as nuclear and cytoplasmic shape and size. A technical analyst then goes through algorithm-generated likely candidates and removes hits that are obviously not cells, such as dye aggregates.

Professional Analysis and Interpretation.

All likely candidate CTCs are presented to a hematopathologist for analysis and interpretation through a web based report where the pathologist is able to include or exclude each candidate cell as an HD-CTC. Cells are classified as HD-CTCs if they are cytokeratin positive, CD45 negative, contained an intact DAPI nucleus without identifiable apoptotic changes (blebbing, degenerated appearance) or a disrupted appearance, and are morphologically distinct from surrounding white blood cells (usually a shape based feature, although occasionally purely size based.) They must have cytoplasm that is clearly circumferential and within which the entire nucleus is contained. The cytoplasm may show apoptotic changes such as blebbing and irregular density or mild disruption at the peripheral cytoplasmic boundary, but must not be so disrupted that its association with the nucleus is in question. The images are presented as a digital image, with individual fluorescent channel viewing capability as well as a composite image. Each cell image is annotated with ancillary statistical data regarding relative nuclear size, fluorescent intensities, and comparative fluorescent intensities. Each HD-CTC candidate is presented in a field of view with sufficient surrounding WBCS to allow for contextual comparison between cytomorphologic features of the cell in question versus the background white blood cells.

The HD-CTC assay was specifically developed with the clinical environment in mind as well as the need for early technology innovation and future automation. All laboratory processes follow strict standard operating procedures that have been optimized, tested and validated. Data collection and candidate identification has been automated using specific interfaces that both enable the pathologist's decision making and subsequent tracking of these decisions. Specifications for both complete automation and adaption to routine settings will arise from this early research framework.

Cell Line Experiments.

Four aliquots from the donor (2 ml each) were spiked with varying numbers of SKBR-3 cells to produce four (4) slides with approximately 300, 100, 30 and 10 cancer cells per a slide. The 16 slides were then processed and analyzed by a single operator according to the HD-CTC sample preparation protocol. A single instrument was used to image all 16 slides.

### REFERENCES

[1] Allard W J, Matera J, Miller M C, Repollet M, Connelly M C, Rao C. Tibbe A G, Uhr J W, Terstappen L W, 2004 Tumor cells circulate in the peripheral blood of all major carcinomas but not in healthy subjects or patients with nonmalignant diseases Clin Cancer Res 10 6897-6904
[2] Cristofanilli M, Budd G T, Ellis M J, Stopeck A, Matera J, Miller M C, Reuben J M, Doyle (G V, Allard W J, Terstappen L W, et al 2004 Circulating tumor cells, disease progression, and survival in metastatic breast cancer N Engl J Med 351 781-91
[3] Miller M C, Doyle G V, and Terstappen L W 2010 Significance of Circulating Tumor Cells Detected by the CellSearch System in Patients with Metastatic Breast Colorectal and Prostate Cancer J Oncol. 617421
[4] Nagrath S, Sequist L V, Maheswaran S, Bell D W, Irimia D. Ulkus L, Smith M R, Kwak E L, Digumarthy S, Muzikansky A, et al 2007 Isolation of rare circulating tumour cells in cancer patients by microchip technology Nature 450 1235-1239
[5] Maheswaran S, Sequist L V, Nagrath S. Ulkus L, Brannigan B, Collura C V, Inserra E, Diederichs S, Lafrate A J, Bell D W, et al 2008 Detection of mutations in EGFR in circulating lung-cancer cells N Engl J Med 359 366-377
[6] Sequist L V, Nagrath S, Toner M, Haber D A, Lynch T J 2009 The CTC-chip: an exciting new tool to detect circulating tumor cells in lung cancer patients. J Thorac Oncol 4 281-283
[7] Pantel K, Alix-Panabieres C, and Riethdorf S 2009 Cancer micrometastases Nat Rev Clin Oncol 6 339-351
[8] Alunni-Fabbroni M and Sandri N T 2010 Circulating tumour cells in clinical practice: Methods of detection and possible characterization Methods 50 289-297
[9] Marrinucci D, Bethel K, Luttgen M, Bruce R H, Nieva J. Kuhn P 2009 Circulating tumor cells from well-differentiated lung adenocarcinoma retain cytomorphologic features of primary tumor type Arch Pathol Lab Med 133 1468-71
[10] Marrinucci D, Bethel K, Lazar D, Fisher J, Huynh E, Clark P. Bruce R. Nieva J., Kuhn P 2010 Cytomorphology of Circulating Colorectal Tumor Cells: a small case series J Oncol 861341
[11] Marrinucci D, Bethel K, Bruce R H, Curry D N, Hsieh B, Humphrey M, Krivacic R T, Kroener J, Kroener L, Ladanyi A, et al 2007 Case study of the morphologic variation of circulating tumor cells Hum Pathol 38 514-9
[12] Hsieh H B, Marrinucci D, Bethel K, Curry D N, Humphrey M, Krivacic R T, Kroener J, Kroener L, Ladanyi A. Lazarus N, et al 2006 High speed detection of circulating tumor cells Biosens Bioelectron 21 1893-1899
[13] Coumans F A, Doggen C J, Attard G, de Bono J S, Terstappen L W 2010 All circulating EpCAM+CK+CD45-objects predict overall survival in castration-resistant prostate cancer Ann Oncol 21 1851-1857

All publications and patents cited in this specification are herein incorporated by reference as if each individual publication or patent application were specifically and individually indicated to be incorporated by reference. Although the foregoing embodiments of the invention have been described in some detail by way of illustration and example for purposes of clarity and understanding, it may be readily apparent to those of ordinary skill in the art in light of the teachings of this invention that certain changes and modifications may be made thereto without departing from the spirit or scope of the inventions described herein. Accordingly, the invention is limited only by the following claims.

What is claimed is:

1. A well for assaying cells which is disposed on a surface of a substrate, the well comprising:
a) a planar bottom surface; and
b) a border forming a periphery of the well, the border being adjacent the bottom surface and providing a fluidic seal therebetween,
wherein the well is configured to receive a monolayer of at least 1.5 million cells within the border, and wherein the planar bottom surface of the well has an area of at least 7.0 cm².

2. The well of claim 1, wherein the well is configured to receive a monolayer of at least 2.5 million cells.

3. The well of claim 1, wherein the well is configured to receive a monolayer of at least 3 million cells.

4. The well of claim 1, wherein the planar bottom surface of the well has an area of at least 10.0 cm².

5. The well of claim 1, wherein the planar bottom surface of the well has an area of at least 11.7 cm².

6. The well of claim 1, wherein the well has a perimeter of at least 12.0 cm.

7. The well of claim 1, wherein the well has a perimeter of at least 14.0 cm.

8. The well of claim 1, wherein the well has a perimeter of at least 15.0 cm.

9. The well of claim 1, wherein the substrate comprises glass.

10. The well of claim 1, wherein the substrate is a planar substrate comprising a length, a width and a thickness.

11. The well of claim 1, wherein the length is about 7 to 8 cm and the width is about 2 to 3 cm.

12. The well of claim 10, wherein the thickness is about 5 to 10 mm.

13. The well of claim 1, wherein the well occupies at least 40%, 53%, or 62% of the surface of the substrate.

14. The well of claim 1, wherein the periphery is rectangular and the substrate is rectangular.

15. The well of claim 1, wherein the border is a structural border or a hydrophobic coating which prevents the flow of fluid through the border.

16. The well of claim 15, wherein the border is a structural border comprising glass.

17. The well of claim 1, wherein the well has a perimeter of at least 15 cm and the planar bottom surface of the well has an area of at least 10 cm².

18. The well of claim 1, wherein the substrate comprises a fiduciary marker.

19. The well of claim 1, wherein the well further comprises a cover slip.

20. The well of claim 1, wherein the planar bottom surface comprises a cell adhesive coating.

21. A system for assaying cells comprising:
a) the well of any of claims 1-20;
b) an illumination system;
c) an imaging system;
d) an analysis module comprising functionality for analyzing cell selection criteria; and
e) a user output.

22. The system of claim 21, wherein the illumination and imaging systems comprise a light source, an excitation filter wheel, a mirror, an optical emission filter wheel, a camera, a light field camera, a data storage module, or a combination thereof.

23. The system of claim 22, wherein the light source is a broad spectrum illuminator.

24. The system of claim 21, wherein the analysis module comprises circuitry operatively coupled to a metadata database populated by data analyzed by the analysis module.

25. The system of claim 21, wherein the cell selection criteria are selected from cellular morphology, nuclear area or size, absence or presence of a cellular marker, intensity of a cellular marker, or a combination thereof.

26. The system of claim 25, wherein the cellular marker is a cell surface marker or a nuclear marker.

27. The system of claim 21, further comprising a data management system.

28. The system of claim 27, wherein the data management system comprising a data storage module.

29. A method for performing a cellular assay, comprising:
a) contacting a sample comprising a population of cells with the well of any of claims 1 to 20; and
b) analyzing the population of cells via the system of any of claims 21 to 28, thereby preforming a cellular assay.

30. The method of claim 29, wherein the analysis comprises characterization of cell types within the population of cells.

31. The method of claim 30, wherein analysis comprises detection of cytokeratin, CD45, nuclear area or size, cellular morphology, or a combination thereof.

32. The method of claim 29, wherein the sample is a blood sample.

33. A method of detecting a circulating tumor cell in a sample having a population of cells, comprising:
a) contacting the well of any of claims 1 to 20 with the sample;
b) analyzing the population of cells via the system of any of claims 21 to 28; and
c) detecting a circulating tumor cell via the analysis of (b), thereby detecting a circulating tumor cell in the sample.

34. The method of claim 33, wherein the sample is a blood sample.

35. The method of claim 34, wherein the sample has a volume of about 1, 2, 3, 4, 5, 6, 7, 8, 9 or 10 ml.

36. The method of claim 33, wherein more than 2, 5, 7, 10, 15, 20 or 50 circulating tumor cells are detected per ml of sample.

37. The method of claim 33, wherein the analysis comprises detection of cytokeratin, CD45, nuclear area or size, cellular morphology, or a combination thereof.

38. The method of claim 37, wherein the circulating tumor cell is characterized as being cytokeratin positive, CD45 negative, and comprising an intact non-apoptotic nucleus via DAPI imaging.

39. A method for diagnosing cancer or providing a prognosis for cancer in a subject, comprising:
a) contacting the well of any of claims 1 to 20 with a sample comprising a population of cells from the subject;
b) analyzing the population of cells via the system of any of claims 21 to 28;
c) detecting a circulating tumor cell via the analysis of (b);
d) characterizing the circulating tumor cell; and
e) determining a diagnosis or prognosis via the characterization of (d), thereby diagnosing or providing a prognosis for cancer in the subject.

40. The method of claim 39, wherein the sample is a blood sample.

41. The method of claim 40, wherein the sample has a volume of about 1, 2, 3, 4, 5, 6, 7, 8, 9 or 10 ml.

42. The method of claim 39, wherein more than 2, 5, 7, 10, 15, 20 or 50 circulating tumor cells are detected per ml of sample.

43. The method of claim 39, wherein the analysis comprises detection of cytokeratin, CD45, nuclear area or size, cellular morphology, or a combination thereof.

44. The method of claim 43, wherein the circulating tumor cell is characterized as being cytokeratin positive, CD45 negative, and comprising an intact non-apoptotic nucleus via DAPI imaging.

45. The method of claim 39, wherein characterizing the circulating tumor cell comprises determining the type of cancer from which the cell originated.

46. The method of claim 39, further comprising administering a chemotherapeutic regime to the subject.

47. The method of claim 46, wherein the regime comprising administration of one or more chemotherapeutic agents.

48. A method for determining responsiveness of a subject to a chemotherapeutic regime, comprising:
a) contacting the well of any of claims 1 to 20 with a sample comprising a population of cell from the subject;
b) analyzing the population of cells via the system of any of claims 21 to 28;
c) detecting a circulating tumor cell via the analysis of (b); and
d) characterizing the circulating tumor cell to determine efficacy of administration of a chemotherapeutic agent, thereby determining responsiveness of the subject to the therapeutic regime.

49. A kit comprising:
a) the well of any of claims 1-20;
b) reagents for immunologically determining the presence of cytokeratin or CD45 in a cell; and
c) instructions for utilizing the kit the detect a circulating tumor cell in a sample.

50. The kit of claim 46, wherein the reagents comprise antibodies which specifically bind cytokeratin and CD45.

51. The kit of claim 46, further comprising reagents for performing DAPI staining.
